(19) United States
(12) Patent Application Publication
(10) Pub. No.: US 2024/0252216 A1
(43) Pub. Date: Aug. 1, 2024

(54) Greater Tuberosity Retainer Plate

(71) Applicant: Stryker European Operations Limited, Carrigtwohill (IE)

(72) Inventors: Nils Zander, Eckernförde (DE); Oliver Kleiner, Schwentinental (DE)

(21) Appl. No.: 18/430,217

(22) Filed: Feb. 1, 2024

Related U.S. Application Data
(60) Provisional application No. 63/442,577, filed on Feb. 1, 2023.

(51) Int. Cl. A61B 17/80; A61B 17/72; A61B 17/86

(52) U.S. Cl. CPC A61B 17/8061; A61B 17/7241; A61B 17/8695

(57) ABSTRACT

A plate configured for placement on an outer surface of a bone includes a plate-shaped body comprised of a malleable material and having an outer perimeter, wherein a portion of the outer perimeter defines an open-ended peripheral seat configured to be located beneath a portion of a head of a fastener, wherein the portion of the outer perimeter defining the open-ended peripheral seat has an open end and an opposite terminal end, the terminal end sized to interact with a single fastener. A surgical method includes inserting a fastener into a bone such that a head of the fastener is not in contact with an outer surface of the bone, placing the plate such that the open-ended peripheral seat is disposed beneath a portion of the head of the fastener, and inserting the fastener further into the bone to contact the head of the fastener to the plate.

### CROSS-REFERENCE TO RELATED APPLICATIONS

The present application claims priority to U.S. Application No. 63/442,577 filed Feb. 1, 2023, the disclosure of which is incorporated herein by reference.

### BACKGROUND OF THE INVENTION

The surgical treatment of multi-fragmentary proximal humerus fractures offers various challenges. Besides a demanding reduction, a generally poor bone quality in combination with complex physiological loads often leads to loss of fixation and revision surgeries. In particular, the reliable and sustaining refixation of the greater tuberosity fragment in 3-and 4-part fractures remains challenging.

Beside conservative treatment, lateral angular stable plating and intramedullary nailing systems are considered the treatment of choice for surgical approaches. Both systems—plates and nails—generally lack an elegant solution to securely fix the greater tuberosity fragment. A limited access/fixation of the posterior aspect of the greater tuberosity is considered essential to prevent clinical complication and ensure a good functional outcome. Considering a generally poor bone quality within the proximal humeral region and the fact that proximal humerus fractures are often associated with soft tissue disruptions, further important requirements for fixation implants are desired.

It is accepted clinical practice to use the proximal screws of a nail construct to re-fixate the soft tissues by means of suture wrap/knotted arrangement around the threaded portion below the screw head. While the use of a washer improves the screw-bone-interface (increased surface area with reduced risk of screw head cortex penetration and subsequent fixation loss), they have limited stability/strength in soft-tissue fixation in case of off-centric loads. There is also a risk of washer rotation.

Accordingly, improvements to the systems and methods for treating proximal humerus fractures are desired.

### BRIEF SUMMARY OF THE INVENTION

The invention includes a washer type clip which can be implanted either during or after screw placement prior to final tightening of the proximal locking screws. Various insertion directions for the washers is possible due to the washers having a different pattern/configuration of oblong holes and/or slots interfacing with the locking screws. Besides the interface to the proximal locking screws, the devices include a posterior extension with one or more tabs which include holes to accepts a fixed-angle, variable-angle or non-locking screw for the posterior fixation of the greater tuberosity. The thickness of the washer differs and an in-situ contouring of the washer might be possible.

A variety of sutures holes in various location is available for soft tissue re-fixation. By using at least two screws for washer fixation, a rotation of the washer (by soft tissue pull) will be effectively prevented.

A first aspect of the present invention is a plate configured for placement on an outer surface of a bone, including a plate-shaped body comprised of a malleable material and having an outer perimeter, wherein a portion of the outer perimeter defines an open-ended peripheral seat configured to be located beneath a portion of a head of a fastener, wherein the portion of the outer perimeter defining the open-ended peripheral seat has an open end and an opposite terminal end, the terminal end sized to interact with a single fastener.

In accordance with other embodiments of the first aspect, the portion of the outer perimeter defining the open-ended peripheral seat may have a substantially circular arc of about 180 degrees, the substantially circular arc being sized to interact with a single fastener. The portion of the perimeter that defines the open-ended peripheral seat may further define an open path extending from the peripheral seat to an exterior of the body, the open path having a substantially constant width substantially equal to a maximum diameter of the substantially circular arc. The open path may extend along a straight line and/or a curved line. The open path may be L-shaped.

The body may define a first aperture bounded within the perimeter and configured for accepting a fastener therethrough. The first aperture may be an elongated slot or may be substantially circular. The body may include a central hub, a first extension projecting away from the hub and including the peripheral seat, and a second extension projecting away from the hub and including the first aperture. A second portion of the outer perimeter may define a second open-ended peripheral seat configured to be located beneath a portion of a head of a fastener, wherein the second portion of the outer perimeter defining the second open-ended peripheral seat has an open end and an opposite terminal end, the terminal end sized to interact with a single fastener. The second portion of the outer perimeter defining the second open-ended peripheral seat may have a substantially circular arc of about 180 degrees, the substantially circular arc being sized to interact with a single fastener.

The body may define a suture aperture bounded within the perimeter and configured for accepting a suture therethrough. A maximum diameter of the suture aperture may be smaller than a maximum diameter of the first aperture. The body may have a variable thickness. The body may have a relatively thicker portion through which the first aperture extends, and a relatively thinner portion in the rest of the body.

A plating system for bone fixation, may include an intramedullary nail configured for placement within an intramedullary canal of a bone, the aforementioned plate, and one or more fasteners. Each fastener may be a fixed-angle screw, a variable-angle screw, or a non-locking screw. A first fastener may be in contact with the peripheral seat, and the second fastener may be in contact with the first aperture.

A second aspect of the present invention is a surgical method including inserting a first fastener into a bone such that a head of the first fastener is not in contact with an outer surface of the bone, placing a plate such that an open-ended peripheral seat defined by a portion of an outer perimeter of a body of the plate is disposed beneath a portion of the head of the first fastener, wherein the portion of the outer perimeter defining the open-ended peripheral seat has an open end and an opposite terminal end, the terminal end sized to interact with a single fastener, and inserting the first fastener further into the bone to contact the head of the first fastener to the plate.

In accordance with other embodiments of the second aspect, the step of placing the plate may include moving the plate along an axis and/or within a plane that is substantially perpendicular to a longitudinal axis of the first fastener. The step of placing the plate may include moving the plate such that a plane defined by a body of the plate is substantially perpendicular to a longitudinal axis of the first fastener. The step of placing the plate may include the terminal end of the open-ended peripheral seat contacting the first fastener. The step of placing the plate may include moving the plate within a plane normal to a location of the bone at which the plate is to be disposed.

The method may further include inserting an intramedullary nail within an intramedullary canal of the bone. The method may further include inserting a second fastener through a first aperture bounded within the perimeter of the body and into the bone. The method may further include inserting a second fastener through a second open-ended peripheral seat defined by a second portion of the outer perimeter of the body and into the bone, wherein the second portion of the outer perimeter defining the second open-ended peripheral seat has an open end and an opposite terminal end, the terminal end sized to interact with a single fastener.

The method may further include inserting a second fastener into the bone such that a head of the second fastener is not in contact with the bone, manipulating the plate such that a second open-ended peripheral seat defined by a second portion of the outer perimeter of the body is disposed beneath a portion of the head of the second fastener, wherein the second portion of the outer perimeter defining the second open-ended peripheral seat has an open end and an opposite terminal end, the terminal end sized to interact with a single fastener, and inserting the second fastener further into the bone to contact the head of the second fastener to the plate.

The bone may be a humerus, and the step of inserting the second fastener may include locating the second fastener in a location on the humerus that is posterior to a location at which the first fastener is located. The method may further include reshaping an extension of the plate containing the first aperture to be located at the greater tuberosity of the humerus, before the step of inserting the second fastener.

The method may further include before placing the plate, inserting a second fastener into the bone such that a head of the second fastener is not in contact with the bone, wherein placing the plate further includes manipulating the plate such that a second open-ended peripheral seat defined by a second portion of the outer perimeter of the body is disposed beneath a portion of the head of the second fastener, wherein the second portion of the outer perimeter defining the second open-ended peripheral seat has an open end and an opposite terminal end, the terminal end sized to interact with a single fastener, and inserting the second fastener further into the bone to contact the head of the second fastener to the plate.

The method may further include passing a suture through a bounded suture aperture defined by the body of the plate, and fixing tissue to the bone by anchoring the suture to the bone using a suture anchor. The method may further include reshaping the body of the plate to substantially match the outer surface of the bone.

A third aspect of the present invention is a plating system for bone fixation, including an intramedullary nail configured for placement within an intramedullary canal of a bone, a fastener having a head and a shaft, the shaft having an outer diameter, and a plate configured for placement on an outer surface of a bone, the plate including: a plate-shaped body comprised of a malleable material and having an outer perimeter, wherein a portion of the outer perimeter defines an open-ended peripheral seat configured to be located beneath a portion of the head of the fastener, wherein the portion of the outer perimeter defining the open-ended peripheral seat has a terminal end having a width that is substantially the same as the outer diameter of the shaft of the fastener.

The present application is directed toward of malleable plate that can be used as a washer which can be inserted after initial fixation of a screw. In that way, the screw can be at least partially inserted, and then the washer can be moved into place and bent or reconfigured to the bone surface. One or more subsequent fasteners can then be inserted into bounded apertures within the plate or washer. The washer can be inserted in a nontraditional way, that is, in a horizontal direction to the bone surface as opposed to being inserted directly toward the bone. This is because the plate is able to be maneuvered underneath the head of a bone screw or fastener that has already been inserted. This provides the surgeon the ability to place the washer after establishing the initial screw insertion. The configuration of the washer then gives the user the flexibility to provide additional fixation beneath the screw heads and to correspond the anchored washer to a secure fixation of the greater tuberosity fragment.

### BRIEF DESCRIPTION OF THE DRAWINGS

FIG. 1 is a perspective view of a washer or washer plate connected by fasteners to a humerus, according to one embodiment of the present invention.

FIGS. 1A-1C are views of an outer perimeter, an open-ended peripheral seat, and an outermost edge of the washer or washer plate shown in FIG. 1.

FIG. 2 is a top view of a humerus including a system having the washer or washer plate shown in FIG. 1, several fasteners, and an intramedullary nail.

FIG. 3 is a perspective view of a washer or washer plate connected by fasteners to a humerus, according to another embodiment of the present invention.

FIG. 4 is a perspective view of a washer or washer plate connected by fasteners to a humerus, according to another embodiment of the present invention.

FIG. 5 is a perspective view of a washer or washer plate connected by fasteners to a humerus, according to another embodiment of the present invention.

FIG. 6 is a perspective view of a washer or washer plate connected by fasteners to a humerus, according to another embodiment of the present invention.

FIG. 7 is a perspective view of a washer or washer plate connected by fasteners to a humerus, according to another embodiment of the present invention.

FIG. 8 is a perspective view of a washer or washer plate connected by fasteners to a humerus, according to another embodiment of the present invention.

FIG. 9 is a perspective view of a washer or washer plate connected by fasteners to a humerus, according to another embodiment of the present invention.

FIG. 10 is a perspective view of a washer or washer plate connected by fasteners to a humerus, according to another embodiment of the present invention.

FIG. 11 is a perspective view of a series of fasteners and an intramedullary nail implanted into a humerus.

FIG. 12 is a perspective view of a washer or washer plate connected by fasteners to a humerus, according to another embodiment of the present invention.

FIG. 13 is a top view of a humerus including a system having the washer or washer plate shown in FIG. 12, several fasteners, and an intramedullary nail.

FIG. 14 is a perspective view of a washer or washer plate connected by fasteners to a humerus, according to another embodiment of the present invention.

FIG. 15 is an enlarged view of a bounded aperture of the washer or washer plate shown in FIG. 14.

### DETAILED DESCRIPTION

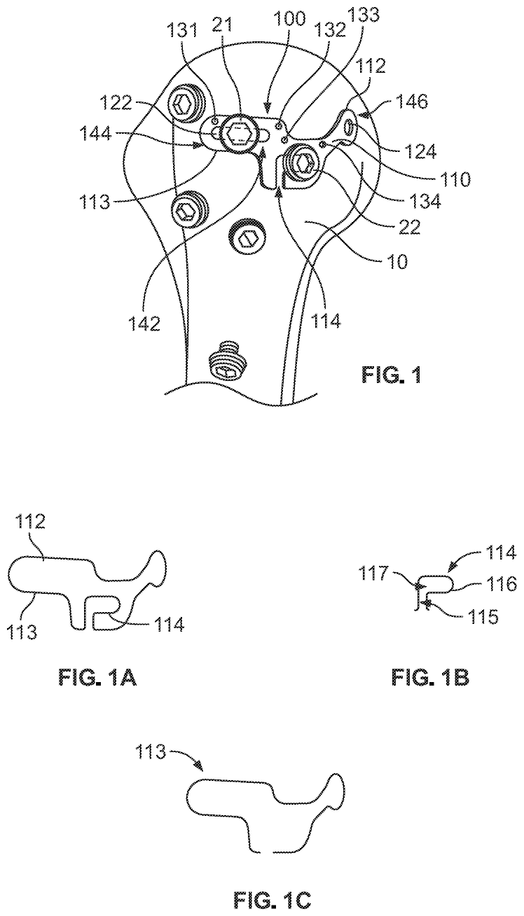

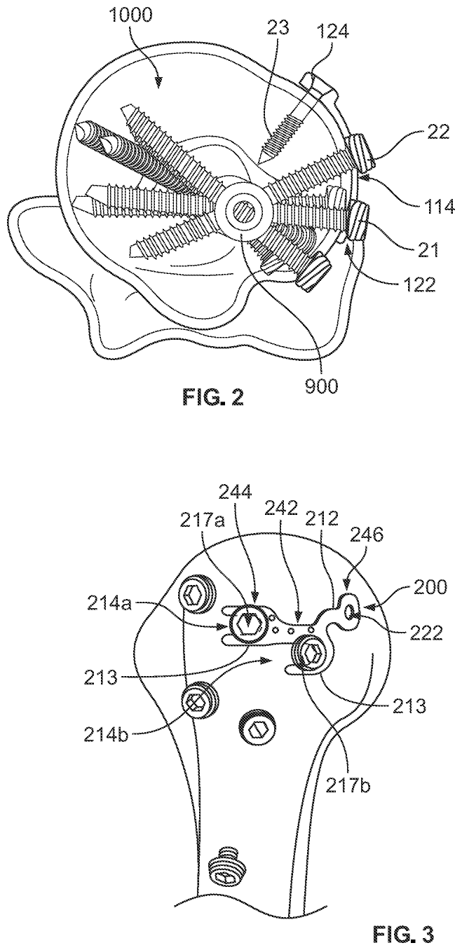

A first embodiment in accordance with the present invention includes a washer or washer plate 100 configured for placement on an outer surface of a bone 10 such as the humerus, as shown in FIGS. 1 and 2. Plate 100 is constructed entirely of a single, monolithic, plate-shaped body 110 that is made of a malleable material such as Titanium Alloy of different Grades, Stainless Steels, other implant materials. Any surgical grade, malleable material is appropriate for use. Plate 100 is configured to interact with fasteners inserted into bone 10, such as fasteners 21 and 22 shown in FIGS. 1 and 2. In this way, plate 100 acts as a washer to provide a more secure connection among the utilized fasteners 21, 22, the bone 10, and an intramedullary nail 900 disposed within the bone. Plate 100 provides more autonomy and flexibility to the user to provide fixation in the most appropriate anatomical locations, including the ability to strategically place fasteners to secure the greater tuberosity fragment and to secure sutures for refixation of surrounding tissue.

Body 110 has a single, continuous outer perimeter 112 that represents an entire outline of body 110, excluding any bounded openings fully encompassed by the material of body 110 and disposed inside this outer perimeter 112. Outer perimeter 112 takes on the form of a single path than can be traced around the entire body 110 without interruption. A silhouette of outer perimeter 112 is shown isolated in FIG. 1A. Outer perimeter 112 is made up of two components: (1) any open-ended peripheral seats for screws or other fasteners, and (2) the remaining outermost edge.

An open-ended peripheral seat 114 that is unbounded at one end is defined by a portion of outer perimeter 112, and is formed as a slot that extends inward from an outermost edge 113 of body 110, as shown in FIG. 1. A silhouette isolating peripheral seat 114 from the remainder of outer perimeter 112 is shown in FIG. 1B. A silhouette isolating the remaining outermost edge 113 from peripheral seat 114 is shown in FIG. 1C. Together, open-ended peripheral seat 114 and outermost edge 113 make up outer perimeter 112. Of course, there may be more than one peripheral seat within outer perimeter 112 as described below.

Outermost edge 113 may be defined as the portion of outer perimeter 112 made up of straight and curved edges excluding relatively sharp bends/turns of about 90 degrees or more. As shown in FIGS. 1 and 1A-1C, open-ended peripheral seat 114 meets outermost edge 113 at two points where outer perimeter 112 changes direction at about a 90-degree angle. Open-ended peripheral seat 114 is a location at which outer perimeter 112 turns back distinctly into body 10 in a direction different or opposite to how outer perimeter 112 would otherwise naturally flow. As shown in FIG. 1A, outer perimeter generally extends in a first direction (e.g., clockwise) from its upper side around the right side, and then open-ended peripheral seat 114 generally extends in an opposite second direction (e.g., counter clockwise).

Open-ended peripheral seat 114 is defined by three portions: (1) an open end or a mouth 115, (2) an opposite terminal end or arced portion 116, and (3) a slot portion 117. Mouth 115 is located where open-ended peripheral seat 114 meets and transitions to outermost edge 113. In other words, mouth 115 is the open space between the adjacent ends of outermost edge 113. Terminal end or arced portion 116 is sized to interact with a single fastener, and has a circular arc of about 180 degrees. That is, terminal end 116 has a width and configuration to mate with a single fastener even though the other dimensions of open-ended peripheral seat 114 may be larger. In some embodiments, arced portion 116 is a semicircular terminal end of peripheral seat 114. Slot portion 117 is of substantially constant width that is substantially equal to a maximum diameter of the circular arced portion 116. Slot portion 117 extends inward into body 110 from mouth 115 to circular arced portion 116. Peripheral seat 114 is designed, dimensioned, and configured to be located beneath a portion of a head of fastener 22, and as such, the width of slot portion 117 is dimensioned slightly larger than a diameter of a screw or fastener shank with which plate 100 is intended to be used. The open path of slot portion 117 and can extend along a straight line or other complex shapes such as a curved line, shown as the open path in FIG. 1 that is also L-shaped. Slot portion 117 may have a constant width along its straight/curved axis, or its width may vary along such axis. In FIG. 1, portions of open-ended peripheral seat 114 are shown in dotted lines to represent those aspects obstructed by fastener 22.

Body 110 also defines a first bounded aperture 122 as an elongated slot that is entirely within outer perimeter 112 and configured for accepting fastener 21 therethrough. Bounded aperture 122 is not open at outer perimeter 112 and is completely closed. A second bounded aperture 124 is also defined within body 110 and is circular. Additional bounded suture apertures 131, 132, 133, 134 are defined within body 110 and configured for accepting respective sutures therethrough. Bounded suture apertures 131-134 are each configured to be smaller than all of the bounded apertures 122, 124 for fasteners, such that a maximum diameter of each suture aperture 131-134 is smaller than a maximum diameter of bounded apertures 122, 124. While depicted with certain numbers of fastener apertures and suture apertures, a plate according to the present invention can have any number of each according to need and configuration.

Body 110 has a variable thickness, as shown in FIG. 2, where it can be seen that body 110 has a relatively thicker portion through which second bounded aperture 124 extends and a relatively thinner portion in the rest of the plate-shaped body 110. This provides a stronger seat for the fastener that anchors the greater tuberosity fragment after the adjacent portion of plate 100 is bent to facilitate alignment of second bounded aperture 124 with the appropriate anatomical location. In other embodiments, the thickness of body 110 may be constant or may have a number of relatively thicker portions at different areas of body 110.

The general layout of body 100 is such that it includes a central hub 142 as the central main portion, and a first extension 144 and a second extension 146 projecting away from hub 142. Peripheral seat 114 is disposed within central hub 142. First extension 144 includes first bounded aperture 122, while second extension 146 includes second bounded aperture 124. As can be appreciated, the design of the plate, and particularly the location and dimensions of extensions protruding outward from central hub 142, can be patient specific or can be variable such that a kit of differently sized and configured plates (such as two or more of plates 100-800) can be provided for use by a surgeon.

Additional embodiments of plates 200-800 in accordance with the present invention are shown in FIGS. 3-9, each being constructed in a similar nature to plate 100 with a single, monolithic, plate-shaped body made of a malleable material. Each plate 200-800 has a single, continuous outer perimeter in the same manner described above. In any of plates 200-800, more or fewer bounded, open-ended, and suture apertures can be provided. Differences among plates 200-800 and with respect to plate 100 are described below, with like reference numerals being similarly numbered.

Plate 200 is shown in FIG. 3. Outer perimeter 212 includes outermost edge 213 interrupted by first open-ended peripheral seat 214a and a second open-ended peripheral seat 214b. To be clear, outermost edge 213 is disposed in two locations labeled in FIG. 3, i.e. between the adjacent seats 214a, 214b on both sides. Slot portions 217a, 217b of respective open-ended peripheral seats 214a, 214b extend along straight lines in plate 200. Bounded aperture 222 is also disposed in plate 200. Here, first extension 244 includes peripheral seat 214a, while central hub 242 includes peripheral seat 214b and second extension 246 includes bounded aperture 222.

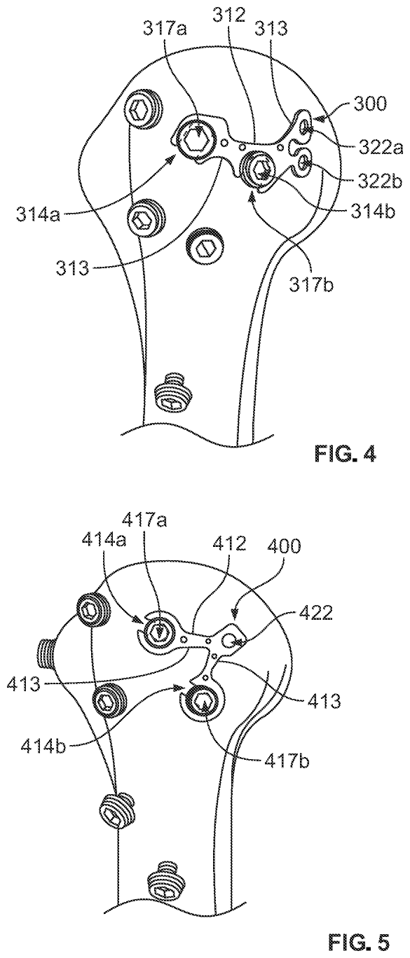

Plate 300 is shown in FIG. 4 with outer perimeter 312 including outermost edge 313 interrupted by first open-ended peripheral seat 314a and second open-ended peripheral seat 314b. Slot portions 317a, 317b of respective open-ended peripheral seats 314a, 314b extend along straight lines in plate 300. First and second bounded apertures 322a, 322b are also disposed in respective extensions of plate 300 from its central hub.

Plate 400 is shown in FIG. 5. Outer perimeter 412 includes outermost edge 413 interrupted by first open-ended peripheral seat 414a and second open-ended peripheral seat 414b. Slot portions 417a, 417b of respective open-ended peripheral seats 414a, 414b extend along straight lines in plate 400. Bounded aperture 422 is disposed in an extension of plate 400.

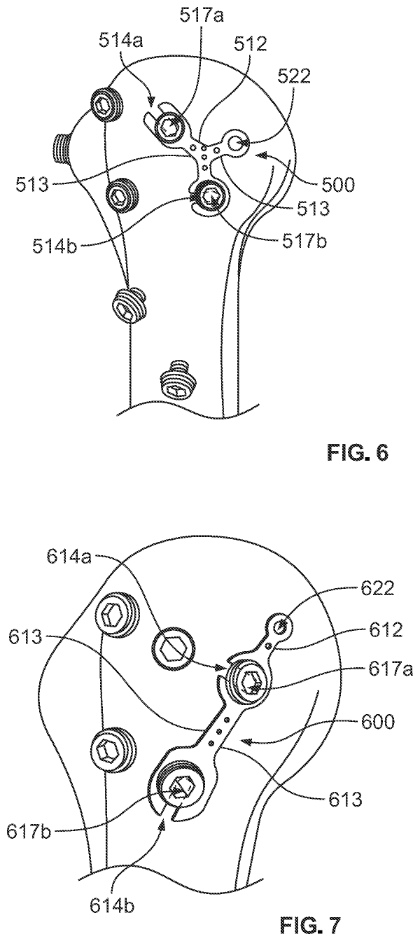

Plate 500 is shown in FIG. 6 and includes outer perimeter 512 with outermost edge 513 interrupted by first open-ended peripheral seat 514a and second open-ended peripheral seat 514b. Slot portions 517a, 517b of respective open-ended peripheral seats 514a, 514b extend along straight lines in plate 500. Bounded aperture 522 is also disposed in plate 500.

Plate 600 is shown in FIG. 7. Outer perimeter 612 includes outermost edge 613 interrupted by first open-ended peripheral seat 614a and second open-ended peripheral seat 614b. Slot portions 617a, 617b of respective open-ended peripheral seats 614a, 614b extend along straight lines in plate 600. Bounded aperture 622 is also disposed in plate 600.

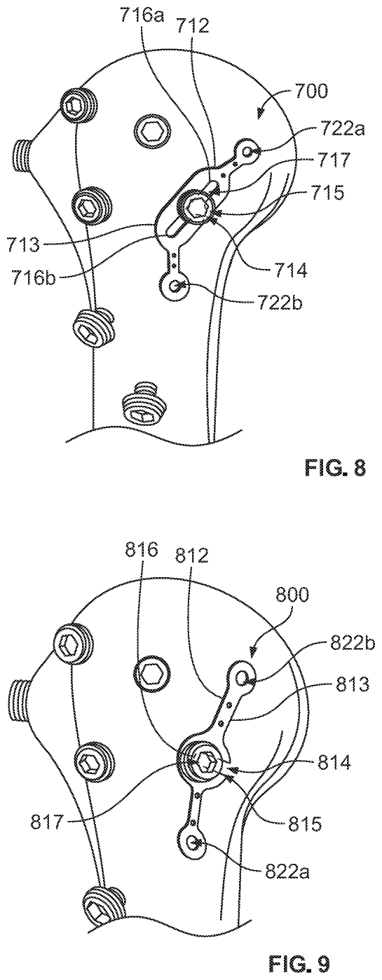

Plate 700 is shown in FIG. 8. Outer perimeter 712 includes outermost edge 713 interrupted by only one open-ended peripheral seat 714. Slot portion 717 extends along a straight line that is generally perpendicular to the direction in which open mouth 715 extends inward from outermost edge 713. Therefore, two arced portions 716a, 716b are disposed at opposite ends of slot portion 717. Two bounded apertures 722a, 722b are also disposed in plate 700.

Plate 800 is shown in FIG. 9. Outer perimeter 812 includes outermost edge 813 interrupted by only one open-ended peripheral seat 814. Slot portion 817 extends along a straight line directly from open mouth 815 to arced portion 816. Two bounded apertures 822a, 822b are also disposed in plate 800.

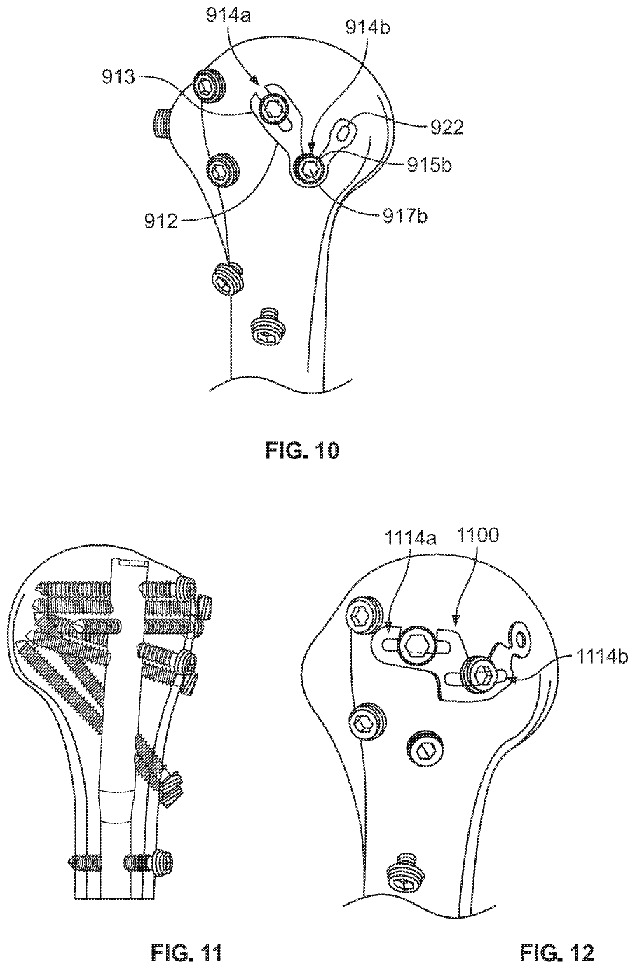

Plate 900 is shown in FIG. 10. Outer perimeter 912 includes outermost edge 913 interrupted by a first open-ended peripheral seat 914a and a second open-ended peripheral seat 914b located inside an elbow of plate 900. Mouth 915b of seat 914b is the opening of seat 914b, where slot portion 917b extending from mouth 915b is of a substantially constant width. In some variations, mouth 915b can have a width slightly less than the substantially constant width of slot portion 917b. In this way, once a fastener is located within seat 914a, plate 900 can be rotated around that fastener until a second fastener clicks into place within seat 914b. Seat 914b defines a location for accommodating a fastener despite that the immediately adjacent portion of outermost edge 913 is not at an angle of around 90 degrees with respect to the linear direction of slot portion 917b. Plate further includes a bounded aperture 922.

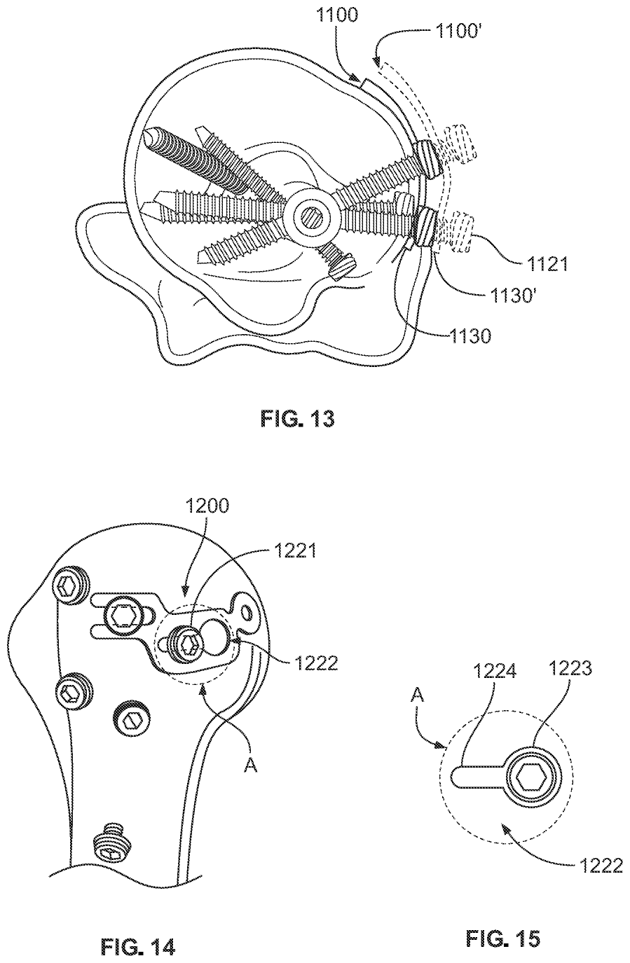

FIGS. 12 and 13 show plate 1100 disposed on a humeral bone. Plate 1100 includes a first open-ended peripheral seat 1114a and a second open-ended peripheral seat 1114b, though either one may instead be a bounded aperture in accordance with the discussion of other variants above. Both seats 1114a and 1114b are elongated slots, which permits for movement of the fasteners within seats 1114a and 1114b as plate 1100 is moved from an initial position and secured to the bone. This initial position of plate 1100 is shown in dashed lines in FIG. 13 (denoted by numeral 1100′), where plate 1100 is initially disposed beneath the heads of the fasteners before the fasteners are fully inserted into their final positions within the bone. Plate 1100 is shown in sold lines in its final positioning against an outer surface of the bone with the fasteners fully seated in their final positions. It is shown that the distance from fastener 1121 to an end 1130′ of plate 1100′ in its initial position is closer than the distance from fastener 1121 to the end 1130 (which is the same) once plate 1100 is fully seated against the bone. Thus, the use of both seats 1114a and 1114b as slots permits the fasteners to move within plate 1100 as necessary based on final seating of plate 1100 against the bone, while maintaining both the seating of plate 1100 in its optimal configuration and orientation as well as the secure fixation of the fasteners with respect to plate 1100 and the bone. FIG. 13 therefore shows plate 1100 in its initial (1100′) and final (1100) implanted stages, along with the trajectories of the fasteners associated with the procedure. FIG. 11 also shows the fasteners in connection with the underlying nail, without plate 1100 for the sake of clarity.

FIG. 14 shows plate 1200 having a keyhole-shaped bounded aperture 1222. Aperture 1222 has an enlarged diameter portion 1223 and a slot portion 1224 which together make up its keyhole shape, as shown further in FIG. 15. In this way, aperture 1222 can be inserted over a head of a preinstalled fastener 1221 having a head of a smaller diameter than the diameter of enlarged diameter portion 1223. Then, plate 1200 can be maneuvered so that the underlying shaft of fastener 1221 is moved into slot portion 1224, thereby permitting fastener 1221 to secure plate 1200 once it is fully inserted into its final position. Other aspects of plate 1200 can be as described in the variations of plates discussed above.

In a further embodiment of the present invention, a plating system 1000 is provided for bone fixation that includes plate 100 with one or more fasteners and an intramedullary nail 900 configured for placement within an intramedullary canal of bone 10. As shown in FIG. 2, fastener 22 is in contact with peripheral seat 114, and fastener 21 is in contact with first bounded aperture 122. Each of the fasteners can be a fixed-angle screw, a variable-angle screw, or a non-locking screw. The system can include two or more fasteners as needed to securely fix plate 100 to bone 10.

A method of using plate 100 and plating system 1000 provides a surgeon with autonomy to first anchor a fastener into a bone before committing to a location at which to place the washer or plate 100. Use of two fasteners with plate 100 also provides an ability to secure sutures to plate 100 for refixation of surrounding tissue without any concern of movement or rotation of plate 100 from its implanted position. The malleable nature of the plate material and the extensions from the central hub of plate 100 also permit securement of the greater tuberosity fracture, such as through fixation within bounded aperture 122.

An initial step of using system 1000 includes inserting nail 900 within an intramedullary canal of bone 10 using known techniques and procedures. The bone can be any long bone, including a humerus, femur, tibia, etc. Once nail 900 is implanted, fastener 22 is inserted into bone 10 such that a head of fastener 22 is not in contact with an outer surface of bone 10. That is, the initial insertion positions fastener 22 to a depth at which its head is not yet in contact with bone 10 as it is driven into place. This insertion may include locating fastener 22 through a bore within nail 900. However, it is also contemplated that a fastener could be secured only within bone and not within nail 900. Ultimately, fastener 22 is partially inserted but not fully seated within the bone, such that a gap exists between its head and bone 10. In this position, the head of fastener 22 is not flush against the surface of bone 10.

Next, plate 100 is placed such that open-ended peripheral seat 114 is disposed beneath a portion of the head of fastener 22. In this way, plate 100 can be positioned between bone 10 and the head of fastener 22 and maneuvered so that a shank of fastener 22 enters mouth 115 and slides along slot portion 117 to a final position at or adjacent to arced portion 116 of open-ended peripheral seat 114. This allows the surgeon to rotate and manipulate plate 100 to seat it in the proper position with respect to fastener 22 and/or nail 900, taking into account the surfaces of bone 10 and any fractures therein. During this step, plate 100 is moved along an axis and/or within a plane that is substantially perpendicular to a longitudinal axis of fastener 22. In other words, a plane defined by body 110 is substantially perpendicular to a longitudinal axis of fastener 22. In this way, plate 100 is moved horizontally or within a plane normal to a location of the bone at which plate 100 is to be disposed until terminal end 116 of open-ended peripheral seat 114 contacts fastener 22. This is substantially perpendicular to the direction along which cortical plates are normally inserted, which is directly toward the bone surface.

A second fastener, fastener 21, is inserted through bounded aperture 122 and into the bone. The oval or elongated nature of aperture 122 provides an ability to maneuver plate 100 for alignment of fastener with a bore in nail 900. When using other versions of the plate such as plate 200, this step can include fastener 21 being inserted through a second open-ended peripheral seat 214a and into the bone. The step of inserting fastener 21 can also be done so that its head is initially not in contact with bone 10 or plate 100.

In other embodiments that include two open-ended apertures such as plate 200, the plate can be manipulated to secure both open-ended peripherals seats 214a, 214b into communication with fasteners that are already secured to the underlying bone and possibly nail 900. Thus, fastener 21 can be inserted in the same manner as fastener 22 before plate 100 is placed against bone 10. Particularly in the case of plate 200, plate 200 can be further manipulated such that peripheral seat 214a is disposed beneath a portion of the head of fastener 21.

With both fasteners 21, 22 secured in the bone and plate 100 or 200, for example, located so that each fastener is properly aligned with a bounded or open-ended aperture, fasteners 21, 22 can be inserted further into bone 10 so that the head of each contacts plate 100, thus fully seating fasteners 21, 22. In other embodiments, insertion of fastener 21 can be done either before or after fastener 22 is further inserted to be flush against plate 100.

When bone 10 is a humerus as shown in FIGS. 1 and 2, a fastener 23 can be placed in bounded aperture 124, which permits fastener 23 to be in a location on the humerus that is posterior to a location at which any other fasteners are located. During such insertion, second extension 146 can be reshaped with respect to the rest of plate 100 due to the malleable nature of its material, so that bounded aperture 124 and the associated fastener can be located at the greater tuberosity of the humerus. This can be done prior to insertion of fastener 23 through bounded aperture 124 so that fastener 23 can be inserted directly into the desired location. With at least one other fastener, and preferably two other fasteners, secured in plate 100 already, this fully utilizes the malleability of plate 100 to provide secure fixation of the greater tuberosity fragment in the desired location.

Once plate 100 is seated properly, an optional step can include passing one or more sutures through any one or more of suture aperture 131-134 to fix or re-fix tissue to bone 100 by anchoring the suture(s) to bone 100 using a suture anchor. Because plate 100 is fixed to the bone with two or more fasteners, rotation of plate 100 on the surface of bone 10 is entirely avoided, providing secure and accurate placement of sutures for proper healing of the soft tissue around the fractured bone.

Ultimately, plate 100 is used after placement of nail 900 and during insertion and/or at final tightening of fasteners 21, 22, which are proximal and/or lateral locking screws. Fastener 23 is a posterior screw that can be locking or non-locking, and is inserted after fasteners 21 and 22 are seated.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A plate configured for placement on an outer surface of a bone, comprising:
a plate-shaped body comprised of a malleable material and having an outer perimeter, wherein a portion of the outer perimeter defines an open-ended peripheral seat configured to be located beneath a portion of a head of a fastener,
wherein the portion of the outer perimeter defining the open-ended peripheral seat has an open end and an opposite terminal end, the terminal end sized to interact with a single fastener.

2. The plate of claim 1, wherein the portion of the outer perimeter defining the open-ended peripheral seat has a substantially circular arc of substantially 180 degrees, the substantially circular arc being sized to interact with a single fastener.

3. The plate of claim 2, wherein the portion of the outer perimeter that defines the open-ended peripheral seat further defines an open path extending from the terminal end to an exterior of the body, the open path having a substantially constant width substantially equal to a maximum diameter of the substantially circular arc.

4. The plate of claim 3, wherein the open path extends along a straight line, along a curved line, and/or is L-shaped.

5. The plate of claim 1, wherein the body defines a first aperture bounded within the perimeter and configured for accepting a fastener therethrough.

6. The plate of claim 5, wherein the first aperture is an elongated slot.

7. The plate of claim 5, wherein the body includes a central hub, a first extension projecting away from the hub and including the peripheral seat, and a second extension projecting away from the hub and including the first aperture.

8. The plate of claim 1, wherein a second portion of the outer perimeter defines a second open-ended peripheral seat configured to be located beneath a portion of a head of a fastener, wherein the second portion of the outer perimeter defining the second open-ended peripheral seat has an open end and an opposite terminal end sized to interact with a single fastener.

9. The plate of claim 5, wherein the body defines a suture aperture bounded within the perimeter and configured for accepting a suture therethrough, and a maximum diameter of the suture aperture is smaller than a maximum diameter of the first aperture.

10. The plate of claim 5, wherein the body has a relatively thicker portion through which the first aperture extends, and a relatively thinner portion in the rest of the body.

11. A plating system for bone fixation, comprising:
an intramedullary nail configured for placement within an intramedullary canal of a bone;
the plate of claim 1; and
a fastener.

12. The plating system of claim 11, wherein the fastener is in contact with the peripheral seat, and further comprising a second fastener in contact with the first aperture.

13. A surgical method comprising:
inserting a first fastener into a bone to a depth at which a head of the first fastener is not in contact with an outer surface of the bone;
placing an open-ended peripheral seat of a plate between the bone and a portion of the head of the first fastener, wherein the open-ended peripheral seat is defined by a portion of an outer perimeter of a body of the plate, and the portion of the outer perimeter defining the open-ended peripheral seat has an open end and an opposite terminal end, the terminal end sized to interact with a single fastener; and
inserting the first fastener further into the bone to contact the head of the first fastener to the plate.

14. The method of claim 13, wherein the step of placing includes moving the plate along an axis that is substantially perpendicular to a longitudinal axis of the first fastener.

15. The method of claim 13, wherein the step of placing includes the terminal end of the open-ended peripheral seat contacting the first fastener.

16. The method of claim 13, further comprising:
inserting an intramedullary nail within an intramedullary canal of the bone.

17. The method of claim 13, further comprising:
inserting a second fastener through a first aperture bounded within the perimeter of the body and into the bone.

18. The method of claim 13, further comprising:
inserting a second fastener into the bone such that a head of the second fastener is not in contact with the bone;
manipulating the plate such that a second open-ended peripheral seat of the plate is between the bone and a portion of the head of the second fastener, wherein the second open-ended peripheral seat is defined by a second portion of the outer perimeter of the body of the plate, and the second portion of the outer perimeter defining the second open-ended peripheral seat has an open end and an opposite terminal end, the terminal end sized to interact with a single fastener; and
inserting the second fastener further into the bone to contact the head of the second fastener to the plate.

19. The method of claim 17, wherein:
the bone is a humerus; and
the step of inserting the second fastener includes locating the second fastener in a location on the humerus that is posterior to a location at which the first fastener is located.

20. The method of claim 19, further comprising:
reshaping an extension of the plate containing the first aperture to be located at the greater tuberosity of the humerus, before the step of inserting the second fastener.
